(19) United States
(12) Patent Application Publication
(10) Pub. No.: US 2015/0265478 A1
(43) Pub. Date: Sep. 24, 2015

(54) CAST COVER

(71) Applicant: Kim Seelinger, Howell, MI (US)

(72) Inventor: Kim Seelinger, Howell, MI (US)

(21) Appl. No.: 14/220,749

(22) Filed: Mar. 20, 2014

(51) Int. Cl. A61F 15/00

(52) U.S. Cl. CPC A61F 15/004

(57) ABSTRACT

A cast cover is formed in a shape of a sheath including a closed first end, an open second end, and a side opening. The side opening includes a first side edge and a second side edge. At least one first fastening device is coupled to the sheath adjacent the first side edge and at least one second fastening device is coupled to the sheath adjacent the second side edge. The first fastening device is configured to be releaseably coupled to the second fastening device. The first fastening device may be a hook feature such as a button secured to an exterior surface of the sheath. The second fastening device may be a retaining feature adapted to be retained on the first fastening device, such as an elastic loop.

### FIELD OF THE INVENTION

The invention relates to a protective covering for an injured limb, and more specifically to a protective covering allowing for side entry of the injured limb.

### BACKGROUND OF THE INVENTION

Many injuries of a person's limbs, including bone fractures, often require the person suffering the injury to have a plaster or resin-coated cast applied to the injured limb. The cast is formed to be rigid to restrict movement of the injured limb to allow the injured limb to properly heal. In many cases, the cast is relatively large when compared to the limb it covers, causing a wearer of the cast to have difficulty fitting ordinary clothing over the cast. Also, many casts are formed to have a single color, such as white, yellow, green, or pink, for example. These bright colors can be undesirable when a wearer of the cast desires for the cast to be inconspicuous, such as during business functions or other formal events.

One particular problem is evident when a person has a cast placed over at least a portion of the person's hand or arm, as a person's hand is a portion of the person's body that is regularly exposed to the ambient environment in almost any social situation. One situation where a person may not be able to expose his or her cast is when the person is exposed to cold temperatures, such as during participation in winter sports and activities. This problem may be even more relevant if the cast is formed to allow the wearer's fingers to extend outside of the cast, as the fingers are further exposed to the cold temperatures of the outside environment without any protection or insulation. Even the largest gloves or mittens are often unable to fit over such a cast, presenting the wearer of the cast few options for either hiding the cast or protecting the wearer's hand and arm from the conditions of the outside environment.

Cast covers are known in the art, but the majority of cast covers are directed toward preventing moisture from entering the cast during activities such as showering or bathing, causing such cast covers to not be directed toward having a desired appearance when the cast cover is worn outside of the home and during social situations. In some situations, the wearer of a cast cover may desire that the cast cover complement the remainder of the wearer's outfit as a fashionable accessory. In other situations, the wearer of the cast cover may desire that the cast cover sufficiently hide the cast from view or allows the cast cover to blend seamlessly into the remainder of the wearer's outfit, as many people who have suffered such injuries wish to draw attention away from the cast to avoid the cast and the injury that necessitated it to not be a primary topic of conversation. In contrast, moisture resistant cast covers best suited for use during bathing often have a bag-like structure, rendering such cast covers unsuitable for use with the remainder of the wearer's outfit.

Additionally, the known cast covers are typically formed to have a single opening formed at an axial end of the cast cover, causing a wearer of the cast cover to have to pull the cast cover over the entirety of the often oversized cast through the single opening. This process can prove to be difficult and time consuming, especially in situations where the wearer of the cast has only a single hand available to aid in placing the cast cover over the cast. Furthermore, many of the known cast covers have a complicated means of securing the cast cover over the cast, which again can prove especially problematic for a user having only a single hand available.

It would therefore be desirable to produce a cast cover that has a desired appearance while also providing warmth and protection from the outside environment to the limb having the cast cover disposed thereover.

### SUMMARY OF THE INVENTION

Compatible and attuned with the present invention, a protective covering for an injured limb allowing for side-entry into the protective covering has surprisingly been discovered.

In one embodiment of the invention, a cast cover is disclosed having a sheath including a closed first end, an open second end, and a side opening. The side opening has a first side edge and a second side edge, wherein at least one first fastening device is coupled to the sheath adjacent the first side edge and at least one second fastening device is coupled to the sheath adjacent the second side edge. The first fastening device is configured to be releaseably coupled to the second fastening device.

A method of producing the cast cover having the side opening is also disclosed. The method comprises the steps of: providing at least one layer of flexible material having a peripheral edge including a first side edge, a second side edge opposite the first side edge, a first end edge, and a second end edge opposite the first end edge; folding the first side edge over to be aligned with the second side edge; securing a first portion of the first end edge to a second portion of the first end edge; and securing at least one fastening device to the at least one layer of material.

### BRIEF DESCRIPTION OF THE DRAWINGS

The above, as well as other objects and advantages of the invention, will become readily apparent to those skilled in the art from reading the following detailed description of a preferred embodiment of the invention when considered in the light of the accompanying drawings:

FIG. 1 is a rear perspective view of a cast cover according to an embodiment of the invention;

FIG. 2 is a right side elevational view of the cast cover illustrated in FIG. 1;

FIG. 3 is a fragmentary cross-sectional side view of a seam of the cast cover illustrated in FIG. 1;

FIG. 4 is front elevational view of a layered sheet of material used to produce the cast cover illustrated in FIG. 1 following an application of a first stitch;

FIG. 5 is a front elevational view of the layered sheet of material illustrated in FIG. 4 following an application of a second stitch;

FIG. 6 is a front elevational view of the layered sheet of material illustrated in FIGS. 4 and 5 following a folding of the layered sheet and application of a third stitch; and

FIG. 7 is a front elevational view of the cast cover illustrated in FIGS. 4-6 following application of a first fastening device and a second fastening device.

### DETAILED DESCRIPTION OF THE INVENTION

The following detailed description and appended drawings describe and illustrate various embodiments of the invention. The description and drawings serve to enable one skilled in the art to make and use the invention, and are not intended to limit the scope of the invention in any manner. In respect of the methods disclosed, the steps presented are exemplary in nature, and thus, the order of the steps is not necessary or critical.

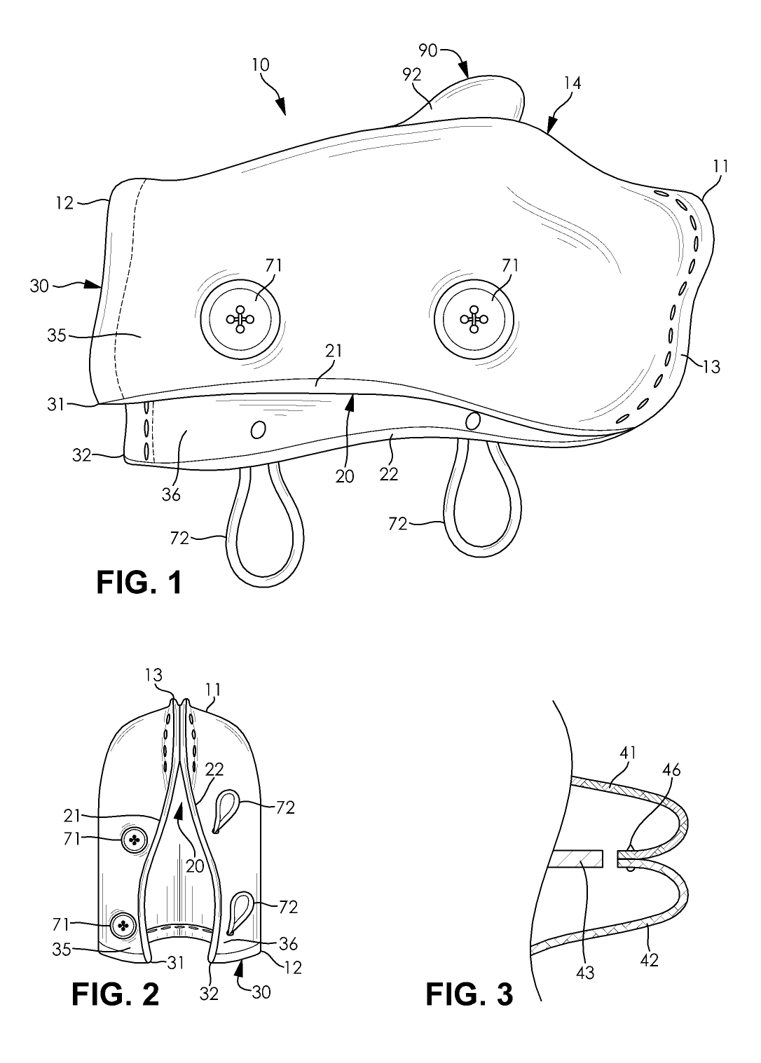

FIG. 1 illustrates a cast cover 10 for covering an injured limb having a cast or other similar protective structure formed thereon according to an embodiment of the invention. The cast cover 10 has a general shape of an elongated sheath including a closed first end 11 and an open second end 12. The cast cover 10 further includes a slit 20 extending from the open second end 12 to the closed first end 11 in a direction along a longitudinal axis of the cast cover 10, the slit 20 providing a side opening for the cast cover 10. At least one first fastening device 71 and at least one second fastening device 72 are coupled to the cast cover 10 adjacent the slit 20.

The open second end 12 of the cast cover 10 includes a lip 30 having a first end 31 and a second end 32. The lip 30 extends circumferentially about the open second end 12 of the cast cover 10 from the first end 31 to the second end 32 thereof. A first side edge 21 of the side opening extends from the first end 31 of the lip 30 in a direction substantially perpendicular to the lip 30 and toward the closed first end 11 and a second side edge 22 of the side opening extends from the second end 32 of the lip 30 in a direction substantially perpendicular to the lip 30 and toward the closed first end 11. The first side edge 21 and the second side edge 22 meet at or adjacent the closed first end 11 of the cast cover 10 to form the slit 20. The slit 20 extends in a longitudinal direction of the cast cover 10 and causes a first flap 35 and a second flap 36 to be formed in the cast cover 10. The first flap 35 may be formed in the cast cover 10 between the first side edge 21 and a portion of the lip 30 adjacent the first end 31 thereof while the second flap 36 may be formed in the cast cover 10 between the second side edge 22 and a portion of the lip 30 adjacent the second end 32 thereof. Because the cast cover 10 is formed from a flexible material such as a fabric, as explained hereinafter, the first flap 35 may be pulled away from the second flap 36 adjacent the open second end 12 of the cast cover 10, causing the slit 20 to have a substantially triangular shape while also widening the opening formed by the lip 30 at the open second end 12 of the cast cover 10 as well as the side opening formed by the slit 20 itself, as best shown in FIG. 2.

The closed first end 11 of the cast cover 10 may have an arcuate profile to cause the first end 11 of the cast cover 10 to resemble a mitten or oven mitt in shape. The closed first end 11 may include a seam 13 extending from a point where the first side edge 21 of the slit 20 meets the second side edge 22 of the slit 20 to a point on an opposite side of the cast cover 10 where a fold 14 extends away from the seam 13 and toward the lip 30. The seam 13 may accordingly have an arcuate profile conforming to a profile of the fingers of a person's hand.

At least one first fastening device 71 is coupled to the cast cover 10 adjacent the first side edge 21 of the slit 20 while at least one second fastening device 72 is coupled to the cast cover 10 adjacent the second side edge 22 of the slit 20. As shown in FIG. 1, the cast cover 10 includes two of the first fastening devices 71, where each first fastening device 71 may be a button disposed on and secured to the exterior surface of the cast cover 10 and spaced apart along a length of the slit 20. Although shown as buttons, it should be understood that the first fastening device 71 may be any type of hook feature suited for releaseably coupling to the second fastening device 72, or any other fastening device configured to cooperate with a second fastening device. The cast cover 10 shown also includes two of the second fastening devices 72, where each second fastening device 72 may be an elastic loop extending away from the exterior surface of the cast cover 10 and spaced apart along the length of the slit 20. Each elastic loop may be formed from a loop of a flexible and elastic material. The elastic loops may also extend from an interior surface of the cast cover 10 and extend beyond the second side edge 22 of the slit 20, as desired. Although shown as elastic loops, it should be understood that the second fastening device 72 may be any type of retaining feature suited for being releaseably coupled to the hook feature of the first fastening device 71, or any other fastening device configured to cooperate with a first fastening device. Additional first fastening devices 71 and second fastening devices 72 can be used if desired. Each of the second fastening devices 72 may be spaced apart along the length of the second side edge 22 of the slit 20 to correspond to a first fastening device 71 disposed on the first side edge 21 of the slit 20. Each of the elastic loops forming the second fastening devices 72 should have a length and elasticity capable of extending around the buttons 73 forming the first fastening devices 71 without rupturing the elastic loop or requiring excessive force.

As shown in FIG. 2, the first fastening device 71 is formed to a left side of the slit 20 while the second fastening device 72 is formed to a right side of the slit 20. This configuration may be more suitable for an injured hand or arm on the right side of a person's body. In the right-handed configuration, the second fastening devices 72 in the form of the elastic loops are formed on a palm side of the slit 20 when the cast cover 10 is placed over a right hand of the user while the first fastening devices 71 in the form of the buttons are formed to a back side of the hand. This configuration allows the slit 20 to be to a side of the hand opposite the thumb. In this configuration, the user can easily grasp one of the elastic loops with a single finger of the user's left hand while pulling the elastic loop over a corresponding button, which can be oriented to face toward the user as the elastic loop is being located around the button. As should be understood, a configuration for a left-handed cast cover 10 may involve changing to which side of the slit 20 that the first fastening devices 71 and the second fastening devices 72 are coupled. Accordingly, the configuration for the left-handed cast cover 10 may include the first fastening devices 71 being coupled to the right side of the slit 20 and the second fastening devices 72 being coupled to the left side of the slit 20, in contrast to the configuration shown in FIG. 2 illustrating the right-handed configuration.

The cast cover 10 may also include a thumb pocket 90 extending from an exterior surface of the cast cover 10 adjacent the fold 14 and to a side of the fold 14 having the second fastening devices 72. However, the thumb pocket 90 may alternatively be formed directly on the fold 14 or to a side of the fold 14 having the first fastening devices 71 secured thereto, as desired. As described hereinabove, a position of the thumb pocket 90 may be dependent on whether the cast cover 10 is in a right-handed configuration or a left-handed configuration. The thumb pocket 90 may be suitable for receiving the user's thumb therein for additional protection from the ambient environment, including cold temperatures. The thumb pocket 90 also provides additional functionality to the hand being placed in the cast cover 10, as the thumb pocket 90 allows the user of the cast cover 10 to grasp various articles between the thumb pocket 90 and the remainder of the exterior surface of the cast cover 10. The thumb pocket 90 and a structure thereof are described in greater detail hereinafter when a method of forming the cast cover 10 is disclosed.

In use, a user of the cast cover 10 pulls the first flap 35 of the cast cover 10 away from the second flap 36 of the cast cover to open and widen the slit 20, and hence the side opening, formed to one side of the cast cover 10. The opening of the slit 20 allows the user to place the injured limb having the cast into the cast cover 10 from the side without having to pull the cast cover 10 over the cast in only an axial direction, which can often be difficult or even painful depending on the size of the cast and the injury to the arm or hand of the user. The cast cover 10 may be configured to cover the user's arm from the wrist to the farthest tip of any digit of the user's hand. It should be understood, however, that the cast cover 10 may be adapted to cover any length of a user's arm, including extending past the elbow, as desired. After the user inserts the hand having the cast formed thereon, the user may select to insert their thumb into the thumb pocket 90, as desired. Once the cast is within the cast cover 10, the user then grasps the second fastening device 72, and fastens it to the first fastening device 71. In the embodiment shown, the first fastening device 71 is placed over the second fastening device 72, releaseably coupling the first side edge 21 of the slit 20 to the second side edge 22 of the slit 20. The cast cover 10 is then secured over the cast of the user.

To remove the cast cover 10, the second fastening device 72 is released from the first fastening device 71 to uncouple the first side edge 21 from the second side edge 22, and the slit 20 is widened again to remove the limb having the cast from the interior of the cast cover 10. With the elastic loops as the second fastening devices 72, each elastic loop can be grabbed from an interior surface of each of the elastic loops with one or more fingers of the non-cast hand and expanded elastically until the elastic loop can be pulled over the button, uncoupling the elastic loop from the button. The ease of coupling and uncoupling the fastening devices 71, 72 through use of a single hand or even a single finger makes use of the elastic loops advantageous.

Although the first fastening device 71 has been described as a button and the second fastening device 72 has been described as an elastic loop, it should be understood that any known coupling means may be used to releaseably couple the first side edge 21 of the slit 20 to the second side edge 22 thereof. For instance, the first fastening device 71 and the second fastening device 72 may be a hook-and-loop feature similar to VELCRO® fasteners. Alternatively, the first fastening device 71 may be a projection adapted to snap fit into a corresponding feature of the second fastening device 72. The slit 20 may also include a zipper formed between the first side edge 21 and the second side edge 22, for example.

Referring now to FIGS. 3-7, a structure of the cast cover 10 may better be understood by disclosing a method of manufacturing the cast cover 10. The cast cover 10 includes at least two layers of material selectively joined to one another and formed into a sheath-like shape. FIG. 3 shows a first material 41 and a second material 42 joined together by a stitch 46. The stitch 46 may be formed from any known form of thread, yarn, wire, or other elongated string-like binding material. The first material 41 and the second material 42 may be the same material or they may be different materials. One material which can be used for either of the first material 41 and the second material 42 is felted wool, which provides for warmth when the cast cover 10 is to be worn in an environment having colder temperatures. The first material 41 and the second material 42 may also be formed from a single or double layer of cotton, for example. The first material 41 may have the same color or pattern as the second material 42 or the first material 41 may have a different color or pattern when compared with the second material 42. The cast cover 10 may also include a third material 43 disposed between the first material 41 and the second material 42 in some embodiments. The third material 43 may be stitched together with the first material 41 and the second material 43 or the third material 43 may act as an intermediate layer used as insulation or a moisture resistant layer, for example.

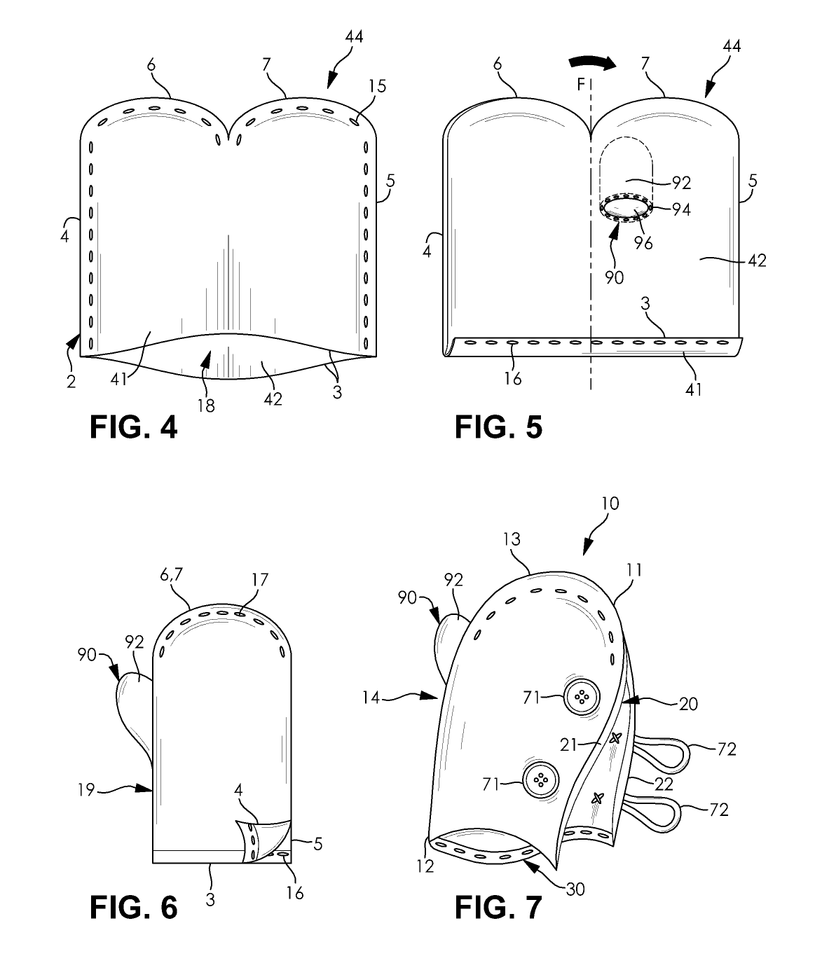

Referring now to FIG. 4, each of the first material 41 and the second material 42 is cut to have a substantially identical size and shape. The first material 41 and the second material 42 are positioned adjacent one another and a peripheral edge of the first material 41 is matched with a peripheral edge of the second material 42, causing the first material 41 to overlay the second material 42.

The overlaid first material 41 and the second material 42 together form a layered sheet 44 having a peripheral edge 2. The peripheral edge 2 includes a first end edge 3, a first longitudinal edge 4, a second longitudinal edge 5, and a second end edge comprising a first arcuate edge 6 and a second arcuate edge 7. The first end edge 3 corresponds to the open second end 12 of the cast cover 10 when the cast cover 10 is completed. The first material 41 is shown as separated from the second material 42 adjacent the first end edge 3 in FIG. 4 to illustrate the distinct layers of material forming the cast cover 10. It should be understood that the first material 41 and the second material 42 may normally be aligned with each other along the first end edge 3 when the overlaying step is performed. The first longitudinal edge 4 extends from a first terminal end of the first end edge 3 in a direction substantially perpendicular to the first end edge 3 and the second longitudinal edge 5 extends from a second terminal end of the first end edge 3 in a direction substantially perpendicular to the first end edge 3. The first arcuate edge 6 extends from an end of the first longitudinal edge 4 opposite the first end edge 3 while the second arcuate edge 7 extends from an end of the second longitudinal edge 5 opposite the first end edge 3.

The first arcuate edge 6 and the second arcuate edge 7 meet at a point substantially aligned with a midpoint of the first end edge 3. The first arcuate edge 6 and the second arcuate edge 7 correspond to the closed first end 11 of the cast cover 10 when the cast cover 10 is fully assembled. The arcuate profile of the first arcuate edge 6 and the second arcuate edge 7 creates a profile at the closed first end 11 corresponding to a profile of a hand to be placed inside the cast cover 10, similar to a shape of a mitten. It should be understood, however, that the first arcuate edge 6 and the second arcuate edge 7 may have any suitable shape for surrounding a hand to be placed within the second closed end 12 of the cast cover 10.

Once the first material 41 and the second material 42 are arranged as shown in FIG. 4, the first longitudinal edge 4, the second longitudinal edge 5, the first arcuate edge 6, and the second arcuate edge 7 are stitched using a first stitch 15 to join the first material 41 to the second material 42. The first stitch 15 may be spaced from the peripheral edge 2 of the laminated sheet 44. Once the first stitch 15 is completed, the first end edge 3 includes a pocket or space 18 formed at the un-stitched portion of the peripheral edge 2 between the first material 41 and the second material 42, as shown in FIG. 4. An interior surface of the layered sheet 44 forming the pocket 18 is then pulled toward the first end edge 3 to turn the laminated sheet 44 inside-out. As a result, the first stitch 15 is hidden within the new interior surface of the laminated sheet 44. Although the laminated sheet 44 has been turned inside-out, it should be understood that the first end edge 3, the first longitudinal edge 4, the second longitudinal edge 5, the first arcuate edge 6, and a second arcuate edge 7 will still maintain the same basic relationship to each other, as shown in FIG. 5. However, in contrast to FIG. 4, the first material 41 is now shown as being behind the second material 42.

Next, the first end edge 3 is folded inwardly toward a side of one of the first material 41 and the second material 42. Whichever material 41, 42 the first end edge 3 is folded toward will represent an interior surface of the completed cast cover 10 adjacent the lip 30 formed in the open second end 12. As shown in FIG. 5, the folded portion of the first end edge 3 is folded toward the second material 42, meaning that the second material 42 will be the material layer contacting the injured limb or cast when the cast cover 10 is being worn. Once folded over, the first end edge 3 is secured to the remainder of the layered sheet 44 by a coupling means such as sewing or stitching. A second stitch 16 may accordingly be formed along a length of the first end edge 3. The fold formed adjacent the now secured linear edge 3 forms the lip 30 between the first longitudinal edge 4 and the second longitudinal edge 5.

If the third material 43 is used, the third material 43 may be inserted between the first material 41 and the second material 42 via the unconnected linear edge 3 prior to the folding and stitching thereof after the layered sheet 44 has been turned inside-out. The third material 43 may be selected depending on a desired characteristic of the cast cover 10. For instance, the third material 43 may be used to insulate the cast cover 10, to prevent excessive moisture from penetrating the cast cover 10, or to add a desired softness or texture to the cast cover 10. The third material 43 may be a thin layer of material such as fabric or the third material 43 may be a plurality of granulate or bead-like structures, as desired.

The thumb pocket 90 may be formed on the cast cover 10 by stitching or otherwise securing a sheath 92 of flexible material to a side of the layered sheet 44 having the first material 41, as shown in FIG. 5, wherein the side of the layered sheet 44 having the first material 41 will eventually represent the exterior surface of the cast cover 10. The sheath 92 may be formed from any flexible material, including but not limited to the first material 41, the second material 42, and the third material 43, as desired. The sheath 92 is elongate and includes an open end for receiving the user's thumb and a closed end formed opposite thereto. As described hereinabove, the thumb pocket 90 may be adapted to receive the thumb of the user therein for additional warmth and protection. Accordingly, the closed end of the sheath 92 may have a substantially hemispherical shape to conform to a shape of the thumb being received therein. The sheath 92 may include a seam (not shown) extending from the open end to the closed end thereof.

Prior to securing the sheath 92 to the layered sheet 44, an aperture 96 may be formed through all of the layers of the layered sheet 44, including the first material 41, the second material 42, and if utilized, the third material 43. The aperture 96 may have a substantially elliptical or circular shape, but it should be understood that any shape for the aperture 96 may be used, as desired. A lip of the sheath 92 forming the open end thereof may then be folded under and inside before being secured to the layered sheet 44 around a periphery of the aperture 96, as shown in FIG. 5. Alternatively, the lip of the sheath 92 may be folded outwardly before the sheath 92 is secured to the layered sheet 44. The sheath 92 may be secured to the layered sheet 44 by applying a pocket stitch 94 around the periphery of the aperture 96. The pocket stitch 94 may extend through the lip of the sheath 92 and at least one layer 41, 42, 43 of the layered sheet 44. For instance, the pocket stitch 94 may extend through the lip of the sheath 92 and the entirety of the layered sheet 44, as shown in FIG. 5.

Once the peripheral edge 2 of the laminated sheet 44 is secured using the first stitch 15 and the second stitch 16, the first longitudinal edge 4 is then folded over to the second longitudinal edge 5 along a fold line F to create a fold 19, as shown in FIGS. 5 and 6. When folded, the first arcuate edge 6 is matched with the corresponding second arcuate edge 7 and stitched thereto by a third stitch 17, forming the closed first end 11 of the cast cover 10. The basic structure of the cast cover 10 is then completed. The stitching of the first arcuate edge 6 to the second arcuate edge 7 causes the first longitudinal edge 4 to meet the second longitudinal edge 5 at the closed first end 11 of the cast cover 10, as shown in FIG. 7. Accordingly, the first longitudinal edge 4 corresponds to the first side edge 21 of the slit 20 while the second longitudinal edge 5 corresponds to the second side edge 22 of the slit 20.

Alternatively, the stitches 15, 16, 17 may be applied to the layered sheet 44 in a second configuration to produce the cast cover 10. Specifically, referring back to FIG. 4, the first stitch 15 may instead be used along the first longitudinal edge 4, the first end edge 3, and the second longitudinal edge 5, leaving the first arcuate edge 6 and the second arcuate edge 7 unstitched. As such, the pocket 18 will be formed between the first material 41 and the second material 42 along the first arcuate edge 6 and the second arcuate edge 7. The interior of the laminated sheet 44 may then be pulled to turn the laminated sheet 44 inside-out, as described hereinabove. Because the first end edge 3 is already stitched, the second stitch 16 may then be applied along the first arcuate edge 6 and the second arcuate edge 7 to close the pocket 18. Alternatively, the second stitch 16 may not be required, as the first longitudinal edge 4 may be folded over the fold line F to the second longitudinal edge 5 to align the first arcuate portion 6 with the second arcuate portion 7, as shown in FIG. 6. When folded, the third stitch 17 may be applied along the matching first and second arcuate edges 6, 7 to combine all of the material layers 41, 42 forming the layered sheet 44 together, eliminating the need for the second stitch 16 to be applied to the first and second arcuate edges 6, 7 prior to the folding step. The addition of the third stitch 17 completes the basic structure of the cast cover 10.

The at least one first fastening device 71 may then be coupled to the layered sheet 44 adjacent the first longitudinal edge 4 and the at least one second fastening device 72 may then be coupled to the layered sheet 44 adjacent the second longitudinal edge 5. If multiple first fastening devices 71 or second fastening devices 72 are used, the fastening devices 71, 72 may be spaced apart along a length of the slit 20. If the button is used, the button may be sewn to be on an exterior surface of the cast cover 10. If the elastic loop is used, the elastic loop may be coupled to the cast cover 10 such that the elastic loop extends away from the exterior surface of the cast cover 10. Alternatively, the elastic loop may extend from an interior surface of the cast cover 10. An end of the elastic loop opposite an end used to surround the button may be disposed to an interior side of the cast cover 10.

The cast cover 10 offers many advantages over the prior art cast coverings. First, the cast cover 10 includes a side opening that allows for side-entry of a limb into the cast cover 10. Second, the cast cover 10 may be formed from materials adapted to insulate the user's hand from cold temperatures while also giving the cast cover 10 an attractive appearance, add durability, any combination of these advantages, or in some circumstances, an inconspicuous appearance. Third, the cast cover 10 includes fastening means that can easily be performed using a single hand or even a single finger, as the elastic loop can be grasped and elastically stretched by placing pressure on any portion of an interior surface of the elastic loop. Finally, the cast cover 10 is easy to manufacture and minimizes a number of parts or materials to produce.

From the foregoing description, one ordinarily skilled in the art can easily ascertain the essential characteristics of this invention and, without departing from the spirit and scope thereof, can make various changes and modifications to the invention to adapt it to various usages and conditions.

What is claimed is:

1. A cast cover comprising:
a sheath including a closed first end, an open second end, and a side opening, the side opening having a first side edge and a second side edge, wherein at least one first fastening device is coupled to the sheath adjacent the first side edge and at least one second fastening device is coupled to the sheath adjacent the second side edge, wherein the first fastening device is configured to be releaseably coupled to the second fastening device.

2. The cast cover according to claim 1, wherein the side opening is a slit extending from the open second end of the sheath to the closed first end thereof, the slit separating the first side edge from the second side edge adjacent the open second end of the sheath.

3. The cast cover according to claim 1, wherein the first side edge of the side opening extends from a first end of a lip forming the open second end of the sheath and toward the closed first end of the sheath and the second side edge of the side opening extends from a second end of the lip forming the open second end of the sheath and toward the closed first end of the sheath, the first side edge meeting the second side edge adjacent the closed first end of the sheath.

4. The cast cover according to claim 1, wherein the first side edge of the side opening cooperates with a first portion of a lip forming the open second end of the sheath to form a first flap and the second side edge of the side opening cooperates with a second portion of the lip forming the open second end of the sheath to form a second flap.

5. The cast cover according to claim 4, wherein the sheath forming the cast cover is formed from a flexible material to facilitate a pulling of the first flap away from the second flap to widen the side opening.

6. The cast cover according to claim 1, wherein the side opening formed in the sheath facilitates side entry of a limb into the cast cover in a direction transverse to a longitudinal axis of the elongated sheath extending from the closed first end to the open second end.

7. The cast cover according to claim 1, wherein the closed first end of the sheath has an arcuate shape.

8. The cast cover according to claim 1, wherein the closed first end of the sheath has a shape conforming to a profile of a human hand.

9. The cast cover according to claim 1, wherein the sheath is formed from at least a first material layer and a second material layer.

10. The cast cover according to claim 9, wherein both the first material and the second material are formed from felted wool.

11. The cast cover according to claim 9, wherein both the first material and the second material are formed from cotton.

12. The cast cover according to claim 1, wherein the first fastening device is a hook feature.

13. The cast cover according to claim 12, wherein the hook feature is a button.

14. The cast cover according to claim 1, wherein the second fastening device is adapted to be retained on a hook feature.

15. The cast cover according to claim 1, wherein the second fastening device is a loop of an elastic material.

16. A method of producing a cast cover comprising the steps of:
providing at least one layer of flexible material having a peripheral edge including a first side edge, a second side edge opposite the first side edge, a first end edge, and a second end edge opposite the first end edge;
folding the first side edge to be aligned with the second side edge;
securing a first portion of the first end edge to a second portion of the first end edge; and
securing at least one fastening device to the at least one layer of material.

17. The method according to claim 16, wherein the first portion of the first end edge is arcuate in shape and the second portion of the first end edge is arcuate in shape.

18. The method according to claim 16, wherein the at least one fastening device includes at least one first fastening device secured to the at least one layer of material adjacent the first side edge and at least one second fastening device secured to the at least one layer of material adjacent the second side edge.

19. The method according to claim 18, wherein the at least one first fastening device is a button secured to the at least one layer of material and the at least one second fastening device is a loop of an elastic material secured to the at least one layer of material.

20. The method according to claim 16, wherein the at least one layer of flexible material includes a first material layer and a second material layer.
